(19) United States
(12) Patent Application Publication
(10) Pub. No.: US 2024/0398501 A1
(43) Pub. Date: Dec. 5, 2024

(54) VOICE AND MOTION CONTROL SYSTEMS AND METHODS FOR SURGICAL EYEWEAR

(71) Applicant: Hawkeye Surgical Lighting, Inc., Coralville, IA (US)

(72) Inventors: David Christianson, Coralville, IA (US); Jeffrey F. Haynes, Clifton, IA (US)

(21) Appl. No.: 18/679,195

(22) Filed: May 30, 2024

Related U.S. Application Data
(60) Provisional application No. 63/469,712, filed on May 30, 2023.
(60) Provisional application No. 63/591,349, filed on Oct. 18, 2023.

(51) Int. Cl. A61B 90/35; A61B 17/00

(52) U.S. Cl. CPC A61B 90/35; A61B 2017/00075; A61B 2017/00203; A61B 2017/00207

(57) ABSTRACT

A lighting system comprising a light source and a handsfree control system in communication with the light source, wherein the handsfree control system is configured for one or more of voice control, motion control, control via biometric feedback, and position-based control of the light source. The lighting system further comprising an accelerometer, a microphone, and/or a surgical navigation system for creating inputs for the system to command control of the light source. The lighting system where the light source is a wearable light for use in an operating theater.

### CROSS-REFERENCE TO RELATED APPLICATION(S)

This application claims the benefit under 35 U.S.C. § 119 (e) to U.S. Provisional Application 63/469,712, filed May 30, 2023, and entitled Voice and Motion Control Systems and Methods for Surgical Eyewear and U.S. Provisional Application 63/591,349, filed Oct. 18, 2023, entitled Surgical Eyewear Lighting Devices, Systems and Methods, each of which are hereby incorporated herein by reference in their entirety for all purposes.

### TECHNICAL FIELD

The disclosure relates to medical/dental lighting systems, and in particular to surgical eyewear lighting systems and the like.

### BACKGROUND

Wearable lamps have become popular for situations in which hands-free use of the lamp is desired. Examples of such situations include surgery and dental procedures, among others. Hands-free situations, such as surgical or dental applications, generally require a human user to engage the patient with fine precision. Thus, adequately illuminating the field of view is an important consideration when selecting a lighting source, such as a wearable lamp.

Dentists and other care providers such as surgeons, doctors, and other professionals may also use loupes. As is generally understood, loupes are magnifying devices that a care provider may wear to improve his or her ability to accurately view the surgical area, such as for example, a patient's anatomy. Some lamps are configured to be fitted on loupes so that the field of view of the surgical site is illuminated and magnified suitably throughout the surgical intervention.

Dentists, surgeons, veterinarians, and others require directed procedural lighting to enable safe completion of dental, medical, and surgical procedures. Practitioners who use surgical telescopes (loupes) to provide magnification of a cavity or wound during procedures often require even more focused procedural lighting in order to ensure complete visualization of the area of interest. It would be understood, that during various procedures, the area of interest may change. Further, the attributes of the area of interest may be significantly different during different portion of the procedure such that one brightness of directed lighting is appropriate for a given area but too bright or too dim for a nearby area. An example of this would be working on the tongue versus working on teeth. The teeth are white and very reflective and as such do not require the same brightness to have the same contrast and definition afforded by the light source. A second example would be during neurosurgical procedures, where the bone of the cranial vault is very bright and superficial and requires very little additional light to provide excellent definition. However, once the bone has been opened and deeper structures entered, the tissues are very quickly less reflective and less clearly defined with the given overhead light requiring additional directed lighting. In cases where the light source is too bright, there can be glare and reflections (so-called washout) that make performing the procedure more difficult.

There is a need in the art for improved devices, systems and methods for illuminating the area of interest in the procedural theater.

### BRIEF SUMMARY

Described herein are various embodiments relating to devices, systems and methods for lighting systems in a surgical, medical and/or dental setting. Although multiple embodiments, including various devices, systems, and methods of the disclosed technology are described herein as a system, this is in no way intended to be restrictive.

In one example, a surgical lighting system comprising a wearable light, and at least one of a motion control system or a voice control system. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In Example 1, a lighting system comprising a light source and a handsfree control system in communication with the light source, wherein the handsfree control system is configured for one or more of voice control, motion control, control via biometric feedback, and position based control of the light source.

Example 2 relates to the light system of any of Examples 1 and 3-12, wherein the light source is a wearable lamp fitted to eyewear.

Example 3 relates to the light system of any of Examples 1-2 and 4-12, further comprising a mobile application or smart watch in communication with the control system wherein one or more commands are programmed via the mobile application for control of the light source.

Example 4 relates to the light system of any of Examples 1-3 and 5-12, further comprising an accelerometer and/or a microphone in communication with a processor of the handsfree control system.

Example 5 relates to the light system of any of Examples 1-4 and 6-12, further comprising a microphone in communication with the handsfree control system, wherein the handsfree control system is configured for voice control, and voice control comprises one or more verbal commands for adjusting the light source to be on, off, brighter, and/or dimmer.

Example 6 relates to the light system of any of Examples 1-5 and 7-12, further comprising an accelerometer in communication with the handsfree control system, wherein the handsfree control system is configured for motion control, and motion control comprises one or more motion commands for adjusting the light source to be on, off, brighter, and/or dimmer.

Example 7 relates to the light system of any of Examples 1-6 and 8-12, wherein the accelerometer is mounted to eyewear to be worn by a user and wherein the one or more motion commands are motions of a head of a user.

Example 8 relates to the light system of any of Examples 1-7 and 9-12, wherein the one or more motion commands comprise a defined deviation from a threshold position of the accelerometer.

Example 9 relates to the light system of any of Examples 1-8 and 10-12, wherein the light source comprising one or more lights for emitting light of different wavelengths, and wherein the handsfree control system is configured for control of the one or more lights via one or more of voice control, motion control, control via biometric feedback, and position based control.

Example 10 relates to the light system of any of Examples 1-9 and 11-12, further comprising a navigation system in communication with the handsfree control system configured to locate a position of an instrument and command changes to the light source based on the position of the instrument.

Example 11 relates to the light system of any of Examples 1-10 and 12, further comprising one or more marker disposed on an instrument configured to locate a position of an instrument and command changes to the light source based on the position of the instrument.

Example 12 relates to the light system of any of Examples 1-11, wherein the handsfree control system controls one or more of an illumination pattern, intensity, wavelength, and on/off status of the light source.

In Example 13, a system for illumination of a surgical field comprising a wearable light comprising a first light source and a second light source and a control system in communication with the wearable light comprising a processor and an accelerometer, wherein the control system is configured for implementing one or more of voice control, motion control, control via biometric feedback, and instrument position based control of the wearable light.

Example 14 relates to the system of any of Examples 13 and 15-20, wherein the control system further comprises a microphone for input of voice commands.

Example 15 relates to the system of any of Examples 13-14 and 16-20, further comprising a surgical navigation system configured for detection of a location of a surgical instrument and commanding the control system to control the wearable light based on the location of the surgical instrument.

Example 16 relates to the system of any of Examples 13-15 and 17-20, further comprising one or more markers on the wearable light or instrument configured for instrument position detection.

Example 17 relates to the system of any of Examples 13-16 and 18-20, wherein the first light source and second light source are of different wavelengths.

Example 18 relates to the system of any of Examples 13-17 and 19-20, wherein the first light source is white light and the second light source is near-ultraviolet light.

Example 19 relates to the system of any of Examples 1-18 and 20, wherein the control system controls one or more of an illumination pattern, intensity, wavelength, and on/off status of the first light source and second light source.

Example 20 relates to the system of any of Examples 13-19, wherein biometric feedback includes one or more of as heart rate, blood oxygenation, respiration rate, end tidal CO2 of respiration, and body temperature.

Example 21 relates to the system of any of Examples 1-20 and 22-23, wherein the handsfree control system or control system is configured to command changes to the light source based on a timing protocol.

Example 22 relates to the system of any of Examples 1-21 and 23, wherein the handsfree control system or control system further comprising a user interface.

Example 23 relates to the system of any of Examples 1-22, further comprising a convolutional neural network.

Other embodiments of the Examples include corresponding computer systems, apparatus, and computer programs recorded on one or more computer storage devices, each configured to perform the actions of the methods.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIGS. 1A is a schematic depiction of the control system in use by a user in a procedural setting, such as an operating room, according to one implementation.

FIGS. 1B is a schematic depiction of the control system in use by a user in a procedural setting, such as an operating room, according to one implementation.

FIGS. 1C is a schematic depiction of the control system in use by a user in a procedural setting, such as an operating room, according to one implementation.

FIGS. 2A is an exemplary depiction of a sensed head position and projection of light output, according to one implementation.

FIGS. 2V is an exemplary depiction of a sensed head position and projection of light output, according to one implementation.

FIG. 3 depicts a user interface for manual adjustment of an aspect such as brightness via a smart phone or other device, according to one implementation.

FIG. 4 depicts a further implementation of a user interface on a watch, according to one implementation.

FIG. 5A is a top view of a PCB, according to one implementation.

FIG. 5B is a bottom view of a PCB, according to one implementation.

FIG. 5C is a side view of a PCB, according to one implementation.

FIG. 5D is a side view of a PCB, according to one implementation.

FIG. 5E is a perspective view of a PCB, according to one implementation.

FIG. 6 is a schematic view of the system in a procedural setting, according to one implementation.

FIG. 7A is a schematic representation of a tissue site for illumination via detection of a position of a surgical instrument, according to one implementation.

FIG. 7B is a schematic representation of a tissue site for illumination via detection of a position of a surgical instrument, according to one implementation.

FIG. 8 is a front view of a wearable light, according to one implementation.

FIG. 9 is a perspective view of a wearable light with markers, according to one implementation.

FIG. 10 is a block diagram of the control system for a wearable light, according to one implementation.

### DETAILED DESCRIPTION

Described herein is a control system for wearable headlamps. The control system may include either or both motion and voice control. The herein described control system for wearable head lamps or lights improves upon the prior art via the use of at least one voice and motion control in a procedural setting, such as a sterile environment. That is, the described system allows for control of the wearable lamp/light via motion or voice control such that a user's hands are not required to control and system and can instead remain sterile and/or be used for actions during the procedure. Various additional use cases and applications are possible and would be appreciated by those of skill in the art in light of this disclosure.

Various further implementations relate to a devices, systems, and methods for controlling wearable headlamps and lighting systems via biometric and/or other peripheral data. It would be understood that the various systems, methods, and devices may be used in combination or separately. That is, in certain implementations, one or more types of controls may be active at any particular time such that the lighting system may be controlled by one or more of motion control, voice control, biometric feedback, and/or feedback from one or more additional peripheral devices, as will be described further herein.

Various implementations of the disclosed technology relate to and/or incorporate various aspects of the teachings of U.S. patent application Ser. No. 18/087,154, filed Dec. 22, 2022, and titled “Surgical Eyewear Lighting Systems and Methods” (“the '154 application”) the content of which is hereby incorporated by reference in its entirety. Some examples of the disclosed technology provide a control system and methods for controlling a wearable head lamp or light used in a procedural setting, such as a sterile environment.

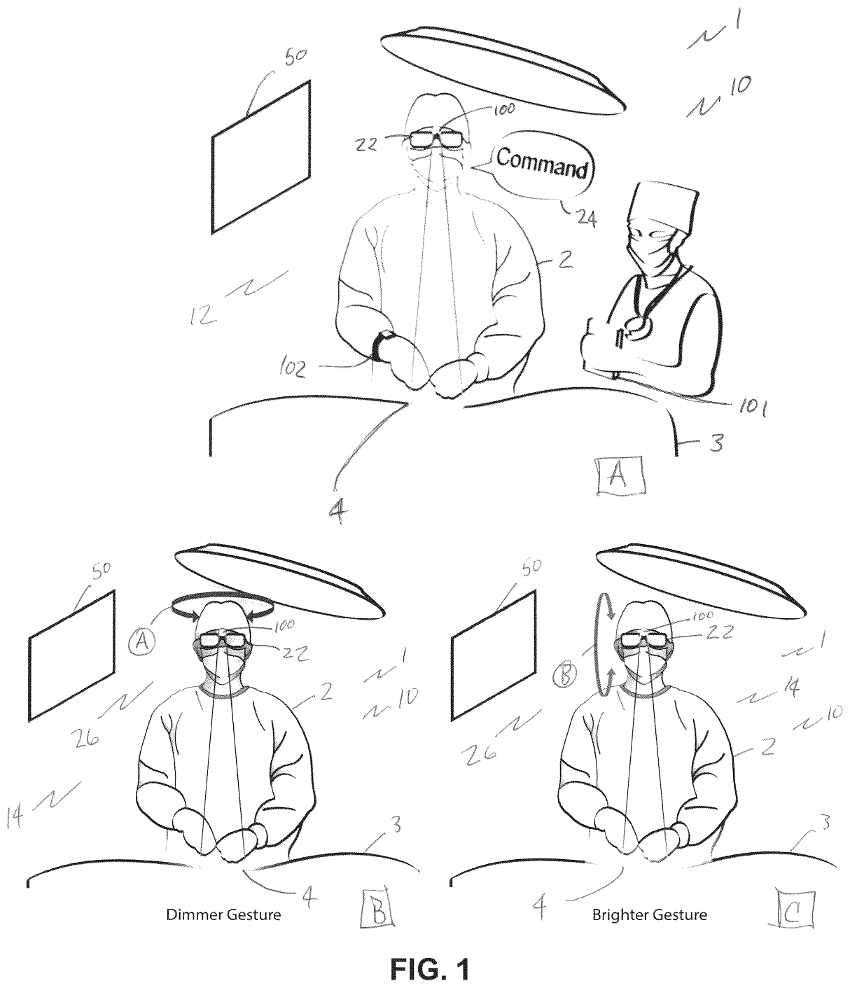

As shown generally in FIGS. 1A-C, certain implementations of the disclosed control system 10 are used in a procedural space 1 such as an operating room for a user 2 such as a physician, dentist, or other healthcare provider treats a patient 3 region of interest 4. In these implementations, the user 2 is utilizing a light 100, such as those implementations incorporate the teachings of U.S. patent application Ser. No. 18/087,154, filed Dec. 22, 2022, which is hereby incorporated by reference in its entirety. Further implementations are usable in other contexts and would be readily appreciated by those of skill in the art.

In various implementations of the system 10, and as shown in FIGS. 1A-C, the light 100 is fitted to a glasses frame 22, as has been described in the incorporated reference. Various implementations of the presently disclosed system utilize voice, motion, or similar control systems to effectuate certain adjustments to the lighting of the region of interest 4 in response to the corresponding actions of the user 2, as would be appreciated. Certain of these implementations allow for the use of an operations unit 50, as well as a mobile device 101, and/or smart watch 102 as optional points of operation, as would be readily appreciated.

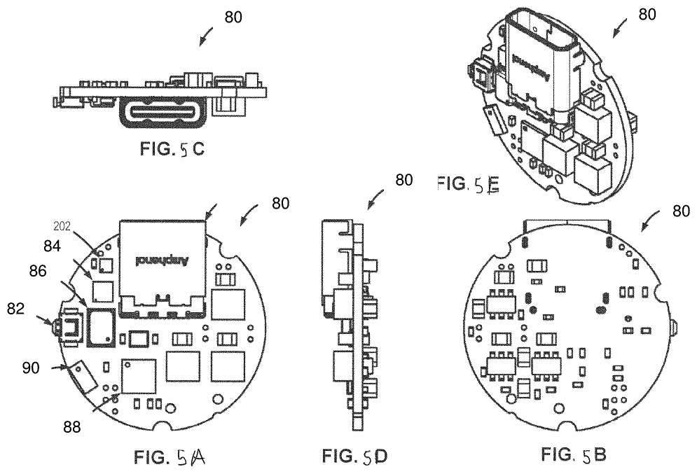

As further shown in the incorporated application, the light 100 according to certain implementations can comprise a printed circuit board (PCB) 80 and various associated components such as an accelerometer 84 and/or microphone 86 disposed on the PCB 80 or elsewhere and in communication with a processor 88 to effectuate the processes described herein. One exemplary implementation is discussed in conjunction with FIGS. 5A-E.

Returning to FIG. 1A, the use of voice control 12 according to certain implementations allows the user to utter a command 24 within a predetermined lexicon, namely by vocalizing understood and defined commands such as “brighter” /“up”, “dimmer” /“down”, or the like to effectuate changes to the light 100 via the control system 10. It is therefore understood that the system 10 utilizes these commands to implement changes to the light brightness by a fixed percentage of total output range each time the command is uttered by the user 4 and recognized by the system 10, such as by way of the microphone 86 or other audio input understood in the art.

As shown in FIGS. 1B-C, further implementations utilize motion control 14, such as via the use of the accelerometer 84. In these implementations, the movement of the user 2, such as the movement of the user's 4 head shown at reference arrows A and B, are detected by the accelerometer 84 and processed by the processor 88 to alter the brightness. There are several functions allowed by such motion control 14, as described further herein.

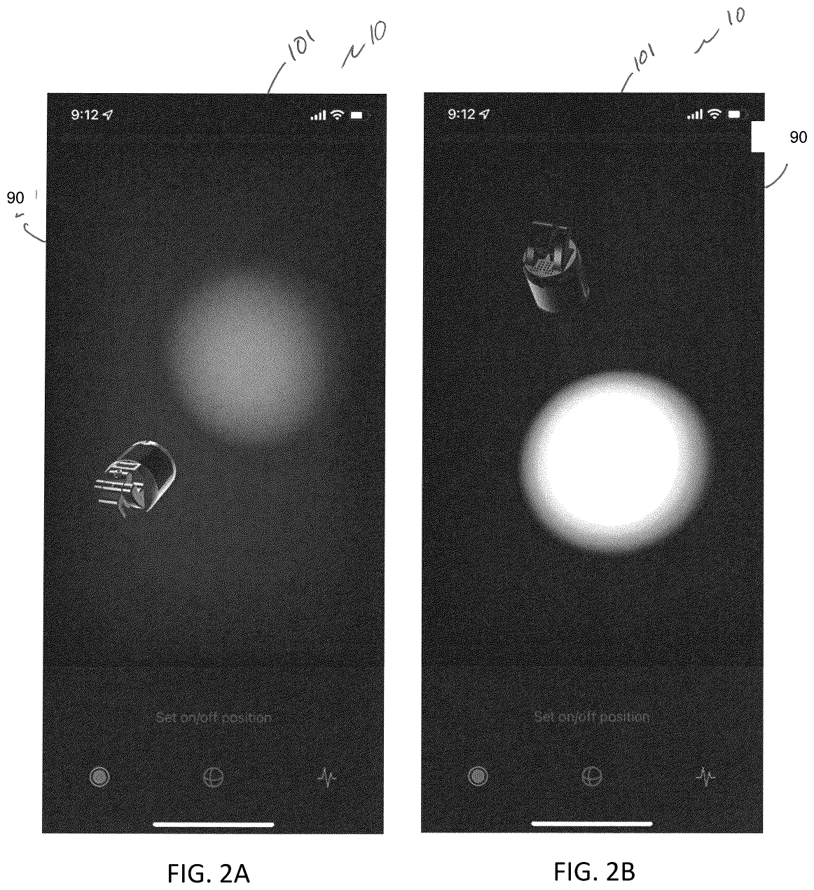

Various implementations of the control system 10 comprise an associated software application (shown, for example at 90 in FIGS. 2A-B), such as a mobile device application such as an iOS or Android application 90, which is optionally Bluetooth connected, as would be understood. In various implementations, the application 90 is run on a control unit 50 and/or on a mobile device 101 and/or smart watch 102, and can be used to take user input to define the various settings and parameters described herein, such as via a graphical user interface (GUI).

The control system 10 may optionally be configured to allow the user to adjust various aspects of the voice 12 and/or motion control 14 systems via the application 90, such as to turn down or shut off the light when the user's 4 head position is above an angle inputted into the system 10 via the application 90, or to define certain voice commands into the lexicon.

In use according to these implementations, the user 4 can place the light 100 on their head and use the application 90 (and subsequently any associated light firmware) to define the threshold position or angle of their head to set the light to be on or off, as would be understood. The system 10, according to these implementations, is tracking the position and space of the light 100 at all times and when conditions are met where the head is above a fixed angle, the light 100 can be dimmed, brightened, turned on, and turned off. It is appreciated that through such use, the user 4 can stand and face someone else and speak to them without blinding them with light. It further allows the user to look at screens to view radiology and other details without glare or washout from the light 100. Further defining an off position may save battery power, so that the light 100 is only in use when it is providing benefit for the procedure.

A further way that motion control 14 is implemented by the system 10 is by allowing gestures to control light output and brightness. Such control allows the user 4 to nod their head or quickly turned their head right or left in a stereotypical way, queuing the system 10 electronics to adjust brightness (i.e., that it should be increased or decreased). Each of these aspects can also be controlled and/or defined via the application 200. In certain implementations, gesturing control can also be used to change light source (discussed separately below).

In various of these implementations, motion control 14 is accomplished by an onboard accelerometer 84 on the main PCB of the headlight that is connected to the microcontroller or processor 88, as shown in FIGS. 5A-E. As would be appreciated, the accelerometer 84 is reporting motion characteristics and/or the angular position of the headlamp 100 at all times, or specified times, to the processor 88.

The processor 88 is correspondingly configured to recognize certain prescribed motions as a command to increase the brightness and/or decrease the brightness, turn the light on or off, and/or change light source. This functionality can be further modified by interfacing with the application 90 which communicates with the headlight and can adjust parameters on the microcontroller to change the response to movement. The user 4 according to these implementations is able to place the light 100 on their head and define stops for where they want the light to turn on and off based on the angular position of the head so that the light will not be on when they are completely upright, such as when they are washing hands, speaking with colleagues, reviewing radiology on screens, and the like. It is further appreciated that in such implementations, the system 10 can be zeroed or otherwise set to default for the individual preferences of several users.

FIGS. 2A-B show examples of sensed head position and projection of light output in use of the system 10 under different operating conditions. In FIG. 2A, the surgeon's head is in an upright position, away from the surgical theater and the system 10 has correspondingly dimmed the light 100 to a low level in conjunction with the detected change in position, such as to about 10% brightness. In FIG. 2B, the surgeon has returned to a downward gaze in the direction of the surgical theater, and in response the system 10 has increased the brightness of the lamp 20 to a high level, such as 90% or 100%. Further implementations are of course possible.

It is appreciated that this motion control 14 system can add additional value in settings where it is too noisy for normal language and speech to be appreciated or if the surgeon does not wish to use voice command to change light settings. Further, it allows for a silent control of light settings that is quick and does not rely on assistance from a helper. Some persons with accents may be self-conscious about using speech for control as they feel that that their pronunciation is not ideal for English language. Alternatively, the system 10 may be programed to recognize key words in various languages in addition to English. In further alternative implementations, the system 10 may be trained on a user or multiple user's voices to learn the needed key words. A motion control 14 approach allows for certain users to feel more confident in their control of the light functions.

A second aspect of the system 10 voice control 12 and motion control 14 is applied in situations involving multi-LED source headlights 100. This allows for a voice command 24, namely “sweep” (or other selected key word) to invoke a change in light source. For example, a specific illustrative use case is provided herein: A cool white LED in the headlight 100 may be outputting at 100%. At a moment in time when the surgeon 4 wants to use a second wavelength light that serves to illuminate tumors or other pathology, invokes the “sweep” command and a prescribed sinusoidal decrease in the cool white light is performed as simultaneously the second wavelength light (such as 410 nanometer near-UV) is ramped up in a corresponding sinusoidal pattern to 100%. In certain implementations, the light pattern remains in this state for a prescribed (though adjustable) period of time, after which a reverse of outputs occurs with resumption of 100% cool white light. It is understood that these settings can be defined through the application 90 in certain situations. Various alternative light patterns are possible and would be appreciated. Further, in certain implementations, the light pattern may remain changed until the user 4 again invokes the “sweep” command and the light pattern reverting to a prior setting.

Such functionality allows a surgeon to have complete contextual lighting for performing the procedure with very specific lighting that highlights pathology or other areas of interest in an intermittent fashion, that can be performed as often as the surgeon wants, without any helpers in the OR switching filters, switching lights, or in any other way relying on external factors. In this way, this described system and devices provide an efficient, sterile, means of switching light sources for the surgeon. Additionally, the system 10 may allow for a reduction of personnel required in the OR, reducing both costs and infection risk.

Similarly, the motion control 14 described above can also have specific gestures assigned to a change of light output so that an appropriate nod or gesture can cause a reduction in cool white light and an intermittent increase in wavelength specific light for visualization of pathology or area of interest. This again can stay for a prescribed amount of time or wait for a second gesture to tell it to return to the initial state, as would be appreciated.

There are several specific examples for use of the system having at least a second wavelength light source, two of which are described here. The first is 405 to 415 nanometer wavelength light or so- called near UV. This is often used to excite a molecule called protoporphyrin 9 which is a metabolite of five aminolaevulinic acid or 5-ALA, which is a medicine given orally to patients before resection of aggressive primary neoplasms of the central nervous system. 5-ALA is selectively taken up by aggressive primary neoplasms of the brain such as glioblastoma, which then convert it to protoporphyrin-IX which glows red when excited by 405 to 415 nanometer light. In this example, the second wavelength of light of interest is 494 nanometers. This wavelength excites a dye called fluorescein, which can be injected by IV into a patient during resections of aggressive tumors. This will cause areas of pathology to emit green light at approximately 520 nanometers. The overall use is the same as for 5-ALA except that it is requiring a different wavelength of light to excite the compound. Control, ultimately, is afforded by two separate LED driver circuits within the main PCB located within the headlight. Through microcontroller pulse width modulation interacting with the LED driver circuit, both LED outputs can be controlled simultaneously.

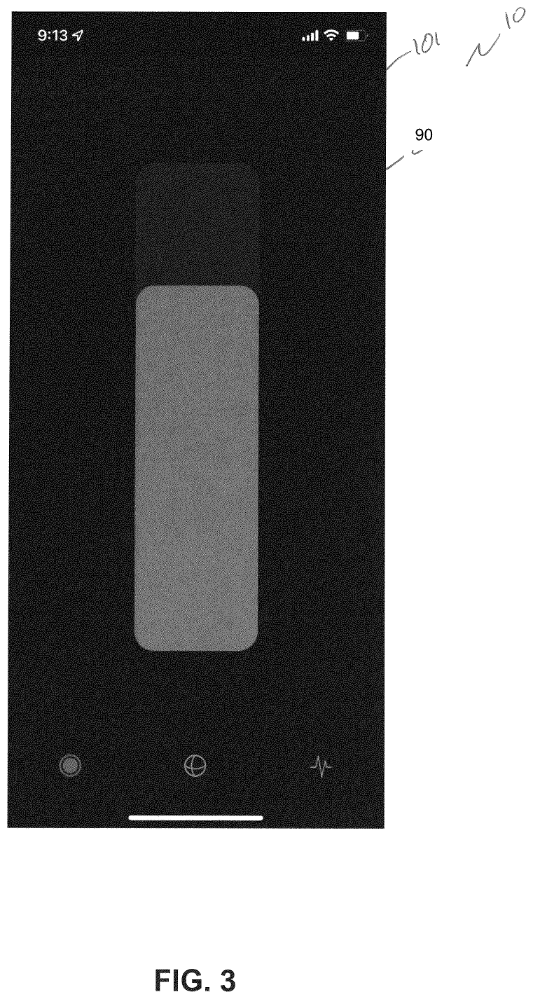

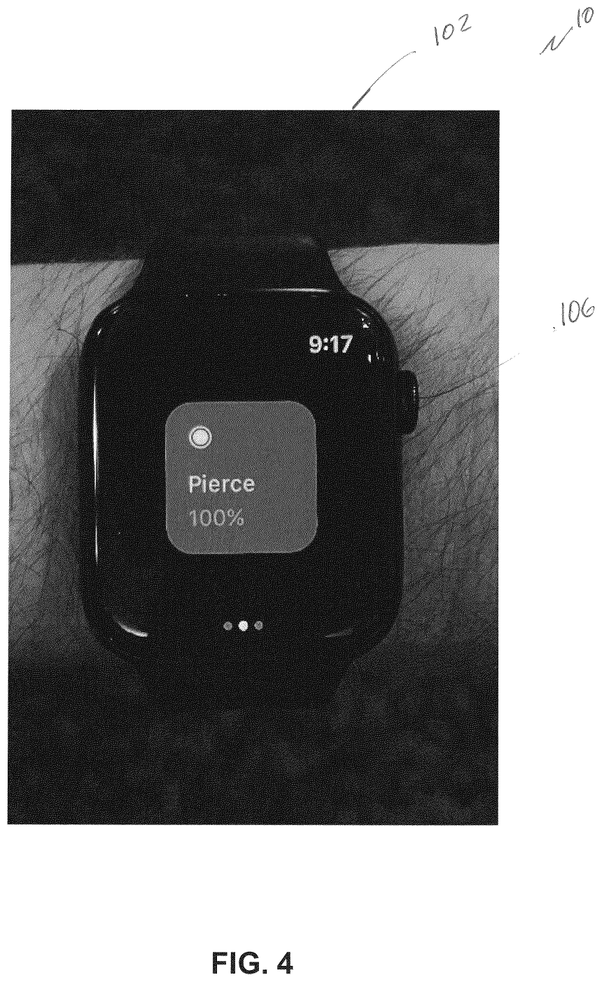

In various implementations, the software application 90 that interfaces with the headlight 100 allows for the graphical depiction of head position and setting of light on and off angles as well as face slider and touch slider control of the light brightness, as is shown in FIG. 3 and can also be implemented in a watch 102 which has functionality, such as to adjust brightness via rotation of the crown 106 control (FIG. 4).

FIGS. 5A through FIG. 5E illustrate various views of a printed circuit board (PCB), in accordance with various implementations. THE PCB 80 may include one or more processors 88, such as is shown at 88. The processor(s) 88 may include one or more controllers (e.g., processors) and one or more tangible, non-transitory memories capable of implementing digital or programmatic logic.

In various implementations, for example, the one or more controllers are one or more of a general purpose processor, digital signal processor (DSP), application specific integrated circuit (ASIC), field programmable gate array (FPGA), a microcontroller, an embedded processor, an application-specific system processors (ASSP), an application-specific instruction set processor (ASIP), an ASIC processor, and/or a multiprocessor, or other programmable logic device, discrete gate, transistor logic, or discrete hardware components, or any various combinations thereof or the like. In various implementations, the processor 88 controls, at least various parts of, and operation of various components of, the surgical light 100. For example, the processor 88 controls various parameters of the LEDs, such as brightness, turning ON and/or OFF, strobe sequences of the LED, switching back and forth between different types of LEDs that emit different frequencies of light, etc. The processor 88 may utilize voice commands and/or sensor input for controlling the LEDs.

The PCB 80 may include an accelerometer 84. The accelerometer 84 may supply surgical light 100 orientation feedback to the processor 88. The processor 88 may be configured to adjust various LED outputs based upon the orientation of surgical light 100 or glasses frame 22. Additionally, accelerometer 84 can be used along with the processor 88 to understand motion commands that can be trained by the user to invoke any and all of the functionality of the surgical light 100. For example, double fast head nod may be trained as a light brighter command, double negative head nod may equal light dimmer, etc. As previously noted, the light on and off may be controlled by movement of the device 100. When the accelerometer 84 detects subtle motion, the processor 88 may control the light to be turned on and if the processor 88 senses no motion for greater than a preset amount of time (e.g., a user definable parameter) the processor 88 may control the light to be turned off.

The processor 88 may utilize accelerometer 84 input for controlling the brightness of the LEDs via processing the orientation and/or angled position of the head of the user 4. More particularly, the processor 88 may be configured to reduce the brightness of the LEDs in response to the accelerometer 84 measuring movement speed and position, as would be understood.

In this manner, the processor 88 may control the surgical light 100 brightness so as to interpret both angle and gestures by the user, that is, by recording both position and acceleration, the system 10 is able to differentiate between different kinds of head movements, as would be appreciated.

The PCB 80 may further include a digital microphone 86. The processor 88 may receive voice commands via digital microphone 86. Various other microphones 86 may be implemented. For example, surgical light 100 may include a stereo microphone arrangement or a microphone array (e.g., for the purpose of beam forming). Various other orientations of the microphone 86 may be implemented. For example, the microphone 86 may be disposed on either side of the PCB 80. Although the sound port for digital microphone 86 is illustrated as facing anteriorly, in various implementations, the sound port may be oriented to face posteriorly.

The PCB 80 may further include ON/OFF switch 82 for turning the surgical light ON or OFF. The PCB 88 may further include an antenna 90.

Various implementations of the system 10 provide real-time feedback to the user 2 during the procedure. By way of a further example, if the user 4 issues a command 24 that the light to get brighter when it is at its full brightness, it blinks twice quickly to let the user know that it is at the maximum range. Similarly, if the user 4 asks for the light to become dimmer and it is at the bottom of its range, the light will blink twice again, signaling to the user 4 that it is at the minimum range. In these and other implementations, the system 10 includes a convoluted neural network (“CNN)” within a microcontroller on the PCB 80 mounted in the back of the surgical headlight for providing feedback to the user 4, such as that shown in FIGS. 5A-E. Various alternative mechanism and algorithms for providing feedback would be recognized by those of skill in the art.

In various implementations, the CNN receives a signal from a digital microphone 86 that is also mounted to the circuit board 80 and pointed towards the operator 4. There is a rolling time window of inferencing that occurs in the microcontroller 80 at all times, looking for the brighter or dimmer command. When the system 10 senses one of these commands with high confidence, it causes the LED output, which is also controlled by the microcontroller 80 to increase or decrease by a fixed percentage, allowing the operator 4 to change brightness without touching anything or without relying on a helper. The benefit to this solution is that it allows the user 4 to change brightness to optimize lighting for all procedural situations while maintaining sterility (they do not need to use their hands to touch a button) and without reliance on a helper in the OR which increases procedure time.

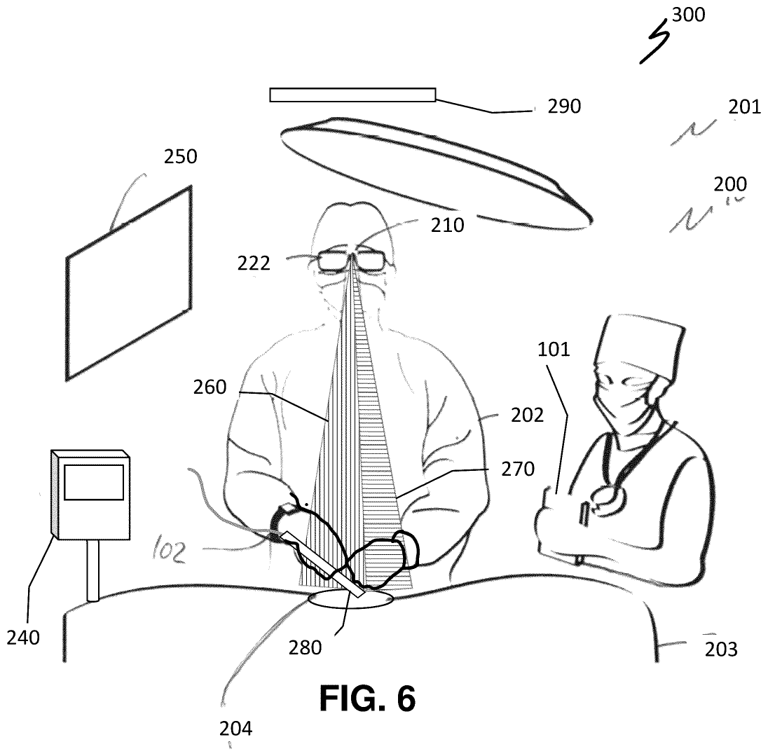

Turning now to FIG. 6, where certain implementations of the disclosed control system 200 are used in a procedural space 201 such as an operating room for a user 202 such as a physician, dentist or other healthcare provider treating a patient 203 region of interest 204, much like the system 100 discussed above. It would be understood that the system 200 of FIGS. 6-10 may be interoperable with the system 100 of FIGS. 1-5E. That is, the system 200 may also include the device, systems, and methods discussed above for voice and motion control. In these implementations, the user 202 is utilizing a wearable lamp or light 210, such as one of the head lights taught in the '154 application and/or as discussed above. Further implementations are usable in other contexts and would be readily appreciated by those of skill in the art.

In various implementations, surgical lighting system 200 includes a wearable light or lamp 210 and a control system 250 or operations unit 250 configured to operate the wearable light 210. In various implementations contemplated herein, and as shown in FIG. 6, the light 210 is fitted to a glasses frame 222 as described in the '154 application. The wearable light 210 may include one or more light sources. As shown in FIG. 6, the wearable light 210 is configured to emit light 260 from a first light source and light 270 from a second light source. Three, four, and more or less light sources may be provided, as would be readily understood.

Certain of these implementations allow for the use of an operations or control unit 250 instead of, or in addition to, a mobile device 101 and/or smart watch 102, discussed above, as optional points of operation, as would be readily appreciated. In various implementations the control system 250 or operations unit 250 may be implemented by circuitry within a separate computing device 240 as shown in FIG. 6. In various cases the control system 250 may be provided by the mobile device 101, smart watch 102, one or more peripheral monitors, or the head light 210 itself.

In various implementations the control system 250 is configured to change the output of the wearable light 210 in response to one or more commands (such as voice or motion commends discussed above), inputs, measurements, stimuli, and/or other conditions. In various cases, the control system 250 is configured to change the output of the wearable light 210 by deactivating the current light source and activating another light source, combining multiple light sources, switching among multiple light sources, changing the intensity of a light source, and changing the timing of a light source, along with various other changes and combinations of changes. Changing the output of the wearable light 210 in various situations allows the surgical lighting system 200 to provide optimal illumination and visualization of the surgical field, surgical site 204, and other areas of interest including, for example, pathologic tissues, as has been discussed further herein.

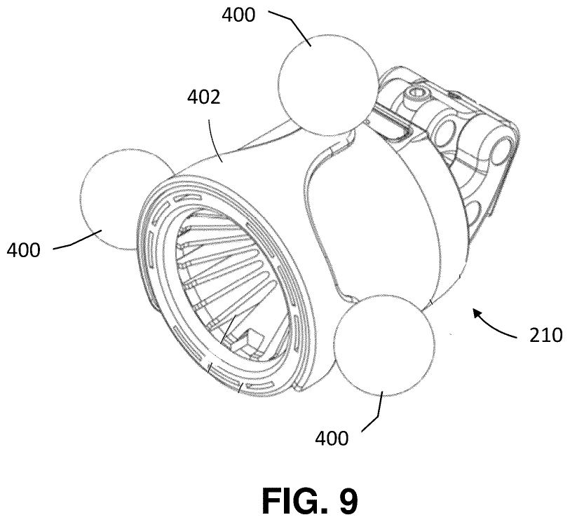

According to various implementations, the surgical lighting system 200 includes and/or is configured to communicate with a surgical navigation system 300. In such cases, the surgical navigation system 300 is configured to indicate the orientation and/or position of one or more items. For example, in various cases the navigation system 300 is configured to indicate the position and/or orientation of the wearable light 210 and/or one or more surgical devices 280 relative to, for example, a patient's anatomy. In some cases, the navigation system 300 includes one or more markers 400 (shown in FIG. 9) placed on the wearable light 210, a surgical device, and/or other items, and a sensor system 290 that detects the presence and location of the markers 400, and thus the location of the surgical device(s) 280.

In various implementations, the lighting 200 and/or navigation system 300 determines that a surgical device 280 is in a certain position (e.g., relative to patient anatomy) and then changes the output of the wearable light 210 accordingly. The systems 200, 300 may further change the light output based on successive positions of the surgical device 280 as indicated by the surgical navigation system 300.

In various implementations the system 200 may change the light output based on the position and/or orientation of the wearable light 210 instead of, or in addition to, the position of one or more surgical devices 280. For example, in some cases the surgical navigation system 300 may determine the position of the wearable light 210 and provide feedback about where the focus of the light is relative to pathologic tissue. In some cases, the system 200 may change the output of the wearable light 210 to indicate what the user 202 is currently focusing on, e.g., a transitional zone or area of interest. In various implementations, the wearable light 210 is registered with the navigation system 300 and receives feedback from the navigation system 300 in order to change the light's output when the center of the area of illumination is in a region that has been pre-identified as an area of tumor, increased contrast uptake, and/or a target identified previously in the navigation system 300. In such cases, the system 200 changes the output of the wearable light 210 (e.g., a change in intensity, wavelength, pulsing frequency, etc.) to indicate to the user/surgeon 202 the relative distance from an area of interest.

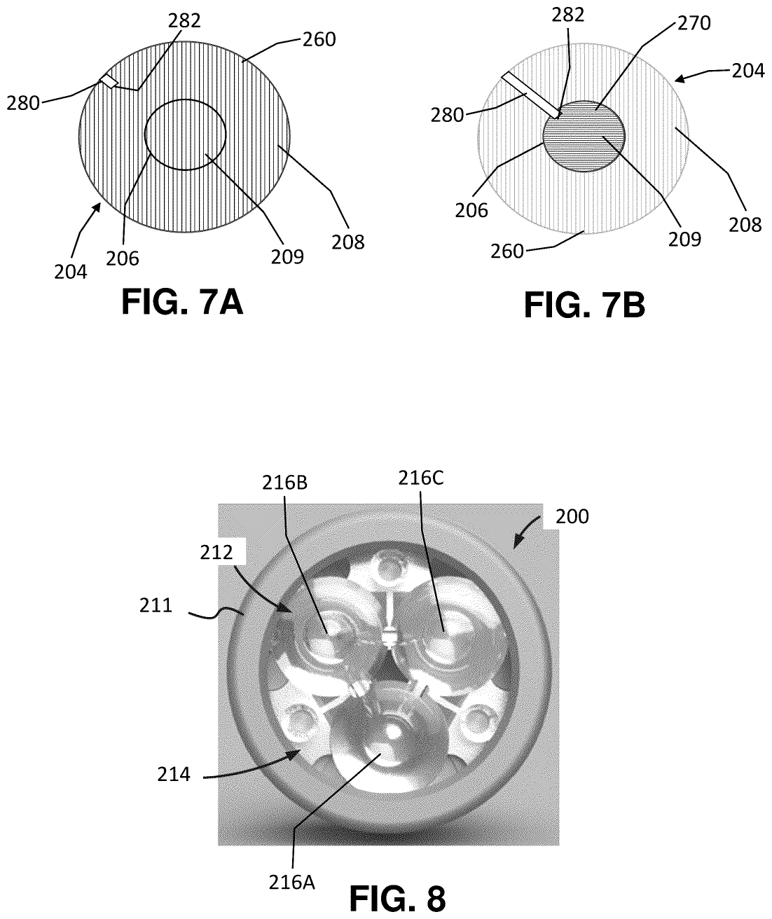

Referring to FIGS. 6 and 7A-7B, according to an example, a suction tube 280 is used for resection of pathologic brain tissue 209 in glioblastoma. In some cases, the navigation system 300 includes markers 400 such as reflective balls (discussed below in relation to FIG. 9) or infrared emitters attached to the suction tube 280 and an optical sensor system 290 installed in the operating room that is able to detect the markers and register the location of the suction tube 280 relative to the patient 203 anatomy. For example, in various cases the navigation system 300 is configured to register the position of the tip 282 of the suction tube 280 and determine when the tip 282 is near the interface 206 between normal brain tissue 208 and identified tumor or abnormal tissue 209 (e.g., as determined through an MRI sequence).

In such cases the control system 250 is configured to recognize when the suction tube 280 is positioned near the interface 206 and change the output of the wearable light 210 in response. As an example, in some cases the control system 250 is configured to decrease the intensity of a first light 260 and increase the intensity of second light 270 emitted from the wearable light 210. The change in lighting can alert the surgeon 202 that the suction tube 280 is near the interface 206 and/or optimize the lighting for. visualizing the difference between normal tissue 208 and abnormal tissue 209. For example, the first light 260 may be used to navigate to the tissue interface 206 and the second light 270 may excite a photosensitive substance to visually distinguish between different tissues.

The system 200 may include various excitation light sources depending upon the characteristics of particular implementations. One example is near-ultraviolet light with a wavelength of about 405 to 415nanometers. In some cases, light with a wavelength of about 410 nanometers is used. In these cases, the control system 250 changes the output of the wearable light 100 by decreasing the intensity of a standard navigation light (e.g., cool white light) and increasing the intensity of near-UV light (e.g., about 410 nm) when the surgical device 280 is near the tissue interface 206.

Continuing with this example, the near-UV light excites a molecule called protoporphyrin 9, which is a metabolite of five aminolaevulinic acid (i.e., 5-ALA), a medicine given orally to patients before resection of aggressive primary neoplasms of the central nervous system. The aggressive primary neoplasms, such as glioblastoma, selectively take up the 5-ALA and convert it to protoporphyrin-IX, which then glows red when excited by 405 to 415 nanometer light.

Another example of an excitation light source illuminates pathologic tissue dyed with fluorescein. In such cases, the fluorescein is introduced by IV into a patient during resections of aggressive tumors. The fluorescein causes areas of pathology to emit green light at about 517 nanometers when excited with light at about 498 nanometers. The effect can, in some cases, make the borders of the tumor fluoresce in the green range in areas that are also associated with margins of the tumor, i.e., areas of increased contrast enhancement in a post contrast T1 MRI. This emission is visible to the human eye and the narrow emission light in the approximately 498 nm excitation/absorption band allows for visible detection of the right shifted light during surgery.

The control system 250 can be configured or programmed to change the output of the wearable light 210 in various manners depending upon the desired illumination pattern, intensity, wavelength, etc. As one example, the control system 250 decreases the intensity of one light source while simultaneously increasing the intensity of another light source. As another example, the control system 250 may rapidly alternate between two separate light sources. Many other changes to the light output are also possible, including changes in light sources, patterns, intensity, and changes to other aspects of the output light.

In various cases the control system 250 may change the output of the wearable light 210 based on one or more patient 203 biometrics. For example, in some cases the control system 250 receives one or more parameters from peripheral devices 240 such as one or more monitors connected to the patient 203. The control system 250 evaluates the parameters and, in some cases, may adjust the output of the wearable light 210 to correspond with the patient 203 parameters/biometrics. As examples, the control system 250 may receive and adjust the light output based on patient 203 biometrics such as heart rate, blood oxygenation, respiration rate, end tidal CO₂ of respiration, body temperature, and the like. In some cases, the control system 250 is configured to time various wavelength intensities with patient 203 biometrics such that the light output changes in time with the patient 203 biometrics. As an example, in some cases the control system 250 may use a patient's heart rate (e.g., specifically systole) to cause a wavelength of light that is preferentially absorbed by oxygenated blood or deoxygenated blood to be pulsed synchronously to help the surgeon/user 202 differentiate between oxygenated arterial vessels and deoxygenated venous vessels.

In various implementations the control system 250 is implemented by a hardware processor along with computer-readable memory programmed with instructions that cause the hardware processor to implement the control system 250. In various cases the control system 250 is implemented by a microcontroller or other suitable hardware processor mounted to a circuit board within the wearable light 210. In such cases the wearable light 210 can include a wireless transceiver that allows the control system 250 to wirelessly communicate with one or more peripheral devices 240 and monitors connected to the patient 203. In some cases, the wearable light 210 also includes temperature sensors, photodiodes (light sensors), current sensors, and/or voltage sensors that also feed data to the control system 250. In these examples the control system 250 receives the various inputs from the on-board devices and/or external peripheral devices 240/monitors and determines how to change the light output according to internal programming including, e.g., one or more algorithms.

Various implementations of the control system 250 comprise an associated software application, such as an application running on a mobile device (e.g., an iOS or Android application). The mobile device 252 may optionally connect to other parts of the system 200 by wireless transmission (e.g., Bluetooth). In various implementations, the application is run on a control system 250 and/or on a mobile device 101 and/or smart watch 102, and can be used to take user input to define the various settings and parameters described herein, such as via a graphical user interface (GUI).

FIG. 8 illustrates a front view of the wearable light 210, in accordance with various implementations of the disclosed technology. This view of the light 210 illustrates the light's bezel 211 and lens 212, as well as an LED star 214 that includes three LEDs in this implementation, while various amounts of LEDs are possible, including one, two, four, or more, as would be understood. In various cases the LED star 214 includes a first LED 216A, a second LED 216B, and a third LED 216C. In various implementations the first LED 216A may comprise an ultraviolet-emitting LED and the second and third LEDs 216B, 216C may produce white light. It should be noted that white LED lights (e.g., the second LED 216B and/or the third LED 216C) may be a cool white LED, or a neutral white LED, or a warm white LED depending on the particular intended application. It should be further noted that the second and third LEDs 216B, 216C may be capable of emitting other color lights (e.g., blue, green, yellow, red, etc.) and that the color of light of the second and third LEDs 216B, 216C is not particularly limited.

In various cases the first LED 216 may be configured to emit ultraviolet (or near ultraviolet) light (also referred to as ultraviolet visible (UVV)). For example, the first LED 216 can be configured to emit ultraviolet light having a wavelength of between 400-420 nm, and in various cases, having a wavelength of between 405-410 nm or 490-505 nm.

In various implementations, the control system 250 is configured to operate the surgical light 210 in one or more manners. For example, the control system 250 may be configured to switch between the first LED 216A, the second LED 216B, and the third LED 216C (e.g., the first LED 216A OFF and the second and the third LEDs 216B, 216C ON, or the first LED 216 ON and the second and third LEDs 216B, 216C OFF, etc.) depending on the system 200 settings and/or a user's preference. Moreover, in some cases the control system 250 may be configured to initiate a strobe sequence back and forth between the first LED 216A and the second and third LEDs 216B, 216C to allow a surgeon 202 to switch between visible white light to view the surgical site 204, and UV light to view pathologic cells with the aid of various compounds (e.g., fluorescent dyes, among others) that act to make pathologic tissue glow or in other ways be visually distinct from normal tissues. For example, in some cases the control system 250 may initiate the strobe sequence when a surgical device (e.g., suction tube) is determined to be positioned near a desired tissue site. Configuring the system 200 to automatically switch among two, three, or more light configurations in this manner enables a surgeon 202 to maintain the field of sterility during a surgical procedure without reaching for dials to adjust light parameters.

FIG. 9 is a perspective view of the wearable light 210 equipped with reflective markers 400 according to various implementations. In various cases the wearable light 210 may also or instead include infrared emitters or another type of navigation aid. In the illustrated example, the reflective markers 400 are part of a navigation collar 402 fitted to the wearable light 210. An example of the navigation collar 402 and reflective markers 400 is illustrated and described in more detail in the '154 application, incorporated herein by reference. According to various implementations, the reflective markers 400 include three spherically shaped balls spaced about 90 degrees apart about the circumference of the navigation collar 402. Other numbers and arrangements of the reflective markers 400 are contemplated. The '154 application also describes an example of calibrating the navigation system 300 using an infrared camera and target that can be used in various cases with the wearable light 210 and the reflective markers 400. According to various implementations, the control system 250 and/or navigation system 300 determines the position of the wearable light 210 using the reflective markers 400. In some cases, the control system 250 may then adjust the light output based on the position of the wearable light 210.

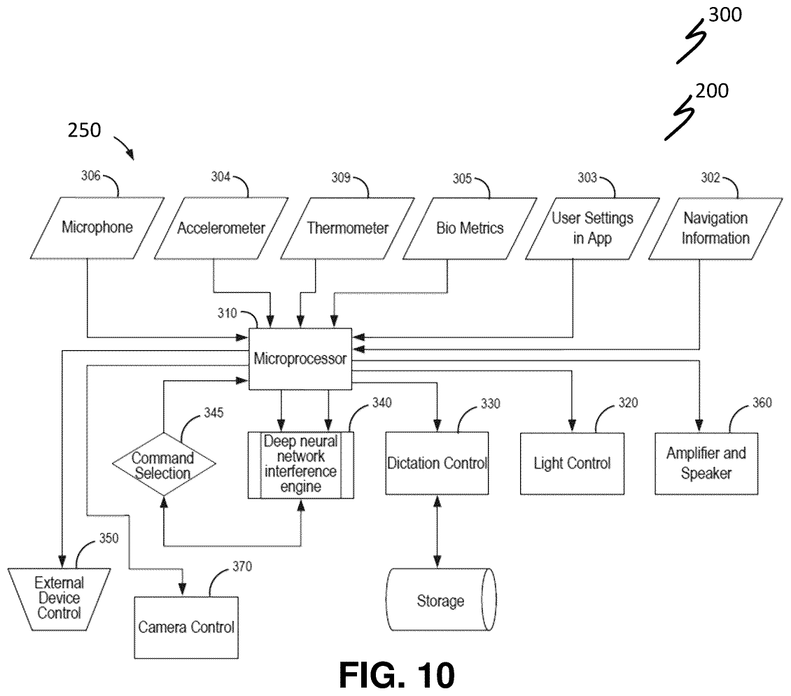

FIG. 10 is a block diagram of a control system 250 for the surgical lighting system 200, in accordance with various implementations. The control system 250 includes a lighting controller 310 (e.g., a processor) configured to receive various input signals, process the inputs according to one or more control algorithms, and send commands to a light control 320 for controlling the state of the LEDs (e.g., ON, OFF, brightness, etc.) of the wearable light 100. As shown in FIG. 10, the control system 250 can include multiple input points that are illustrated and described in more detail with respect to FIGS. 14 and 21 in the '154 application.

According to various implementations, multiple LEDs of different wavelengths can also be controlled in real time by patient-and surgeon-measured characteristics and lab values. As previously discussed, in some cases the control system (e.g., controller 310) can receive biometric signals from one or more biometric sensors 305 associated with a patient and/or a surgeon engaged transducers that are measuring temperature, pulse, blood pressure, oxygen saturation, photoplethysmography, plethysmography, medication and chemistry administration, ventilation parameters, somatosensory evoked potentials, motor evoked potentials, electromyogram, electroencephalography, electrocardiogramand other electrical, mechanical, and chemical sensors. These biometric signals can be processed, interpreted and used to modulate light output characteristics in ways that provide additional insight into the patient's anatomy or in response to changes in surgeon condition. The control system 250 may receive the biometric signals via wireless transmission (e.g., Bluetooth Low Energy (BLE)) and/or wired signal.

In one exemplary implementation, two wavelengths of light that are absorbed differently by oxygenated hemoglobin and deoxygenated hemoglobin may be modulated in a way that the light that is absorbed more completely by oxygenated arterial blood is shone more brightly in a pattern that is temporally associated with the arterial phase of the patient's cardiovascular cycle. Another wavelength of light that is absorbed more completely by deoxygenated blood may be modulated to shine more brightly during the venous phase of the patient's cardiovascular cycle. The differences appreciated may provide additional confidence and insight in differentiating between arterial and venous vessels as well as judging areas of relative ischemia during procedures.

In another exemplary implementation, certain wavelengths of light may be modulated in a timed fashion that is coordinated with the administration of a substance or medication. This may be done to facilitate further visualization of the distribution of said substances or medications which may be selectively taken up by certain groups of cells representing healthy cells or pathologic cells.

According to various implementations the control system 250 can further receive navigation information 302. For example, the navigation system 300 (interfaced with or integrated with the control system 50) may determine the position of the wearable light 210, a surgical device, and/or another instrument and change the output of the wearable light 210 in a desired manner. In various cases the navigation system 300 is configured to determine the position relative to various patient anatomy. The control system 250 may receive the navigation information 302 via wireless transmission (e.g., Bluetooth), or via an application running on a mobile device, such as a smartphone, tablet, or smart watch paired with the wearable light, etc. In some cases, the control system 250 may be implemented on board the wearable light 210. In some cases, the control system 250 may be implemented by a separate control device, such as a computer or a mobile computing device.

Although the disclosure has been described with reference to preferred embodiments, persons skilled in the art will recognize that changes may be made in form and detail without departing from the spirit and scope of the disclosed apparatus, systems and methods.

What is claimed is:

1. A lighting system comprising:
(a) a light source; and
(b) a handsfree control system in communication with the light source,
wherein the handsfree control system is configured for one or more of voice control, motion control, control via biometric feedback, and position-based control of the light source.

2. The light system of claim 1, wherein the light source is a wearable lamp fitted to eyewear.

3. The light system of claim 1, further comprising a mobile application or smart watch in communication with the control system wherein one or more commands are programmed via the mobile application for control of the light source.

4. The light system of claim 1, further comprising an accelerometer and/or a microphone in communication with a processor of the handsfree control system.

5. The light system of claim 1, further comprising a microphone in communication with the handsfree control system, wherein the handsfree control system is configured for voice control, and voice control comprises one or more verbal commands for adjusting the light source to be on, off, brighter, and/or dimmer.

6. The light system of claim 1, further comprising an accelerometer in communication with the handsfree control system, wherein the handsfree control system is configured for motion control, and motion control comprises one or more motion commands for adjusting the light source to be on, off, brighter, and/or dimmer.

7. The light system of claim 6, wherein the accelerometer is mounted to eyewear to be worn by a user and wherein the one or more motion commands are motions of a head of a user.

8. The light system of claim 6, wherein the handsfree control system is configured to command changes to the light source based on a timing protocol.

9. The light system of claim 1, wherein the light source comprising one or more lights for emitting light of different wavelengths, and wherein the handsfree control system is configured for control of the one or more lights via one or more of voice control, motion control, control via biometric feedback, and position-based control.

10. The light system of claim 1, further comprising a navigation system in communication with the handsfree control system configured to locate a position of an instrument and command changes to the light source based on the position of the instrument.

11. The light system of claim 1, further comprising one or more marker disposed on an instrument configured to locate a position of an instrument and command changes to the light source based on the position of the instrument.

12. The light system of claim 1, wherein the handsfree control system controls one or more of an illumination pattern, intensity, wavelength, and on/off status of the light source.

13. A system for illumination of a surgical field comprising:
(a) a wearable light comprising a first light source and a second light source; and
(b) a control system in communication with the wearable light comprising:
(i) a processor; and
(ii) an accelerometer,
wherein the control system is configured for implementing one or more of voice control, motion control, control via biometric feedback, and instrument position based control of the wearable light.

14. The system of claim 13, wherein the control system further comprises a microphone for input of voice commands.

15. The system of claim 13, further comprising a surgical navigation system configured for detection of a location of a surgical instrument and commanding the control system to control the wearable light based on the location of the surgical instrument.

16. The system of claim 13, further comprising one or more markers on the wearable light or instrument configured for instrument position detection.

17. The system of claim 13, wherein the first light source and second light source are of different wavelengths.

18. The system of claim 13, further comprising a convolutional neural network.

19. The system of claim 13, wherein the control system controls one or more of an illumination pattern, intensity, wavelength, and on/off status of the first light source and second light source.

20. The system of claim 13, wherein biometric feedback includes one or more of as heart rate, blood oxygenation, respiration rate, end tidal CO₂ of respiration, and body temperature.
